(19) United States
(12) Patent Application Publication
(10) Pub. No.: US 2025/0376705 A1
(43) Pub. Date: Dec. 11, 2025

(54) PROCESS FOR RECOVERING AND PURIFYING POLYHYDROXYALKANOATES FROM A FERMENTATION BROTH

(71) Applicant: VERSALIS S.P.A., San Donato Milanese (MI) (IT)

(72) Inventors: Daniele RIVA, Rivalta Scrivia (AL) (IT); Tommaso PRANDO, Novara (NO) (IT); Alessandro DEL SEPPIA, Mantova (MN) (IT); Alessandra FRATTINI, Fraz. Rivalta Scrivia (AL) (IT); Paolo MARIANI, Rivalta Scrivia (AL) (IT)

(21) Appl. No.: 18/876,997

(22) PCT Filed: Jun. 26, 2023
(86) PCT No.: PCT/IB2023/056568

§ 371 Date: Dec. 19, 2024

(30) Foreign Application Priority Data
Jun. 27, 2022 (IT) 102022000013483

(51) Int. Cl. C12P 7/625; C08G 63/06

(52) U.S. Cl. CPC C12P 7/625; C08G 63/06

(57) ABSTRACT

A process for recovering and purifying polyhydroxyalkanoate (PHA) from a fermentation broth deriving from the fermentation of microorganisms includes the following steps:

(a) subjecting the fermentation broth as such to heat treatment in the presence of at least one acid;
(b) subjecting the fermentation broth obtained in step (a), directly or after storage, to separation obtaining an aqueous suspension of cellular biomass having polyhydroxyalkanoate (PHA) and an aqueous phase having fermentation residues;
(c) subjecting the aqueous suspension of cellular biomass having polyhydroxyalkanoate (PHA) obtained in step (b) to extraction, purification and drying; wherein step (a) is carried out at a pH between 4 and 6, at a temperature between 50° C. and 70° C., for a time between 5 minutes and 30 minutes.

The aforementioned polyhydroxyalkanoate (PHA) can be advantageously used in various applications, in particular in the medical, pharmacological, cosmetic, agricultural, engineering and food packaging fields.

### CROSS-REFERENCE TO RELATED APPLICATIONS

This application is a 35 U.S.C. § 371 National Stage patent application of PCT/IB2023/056568 filed 26 Jun. 2023, which claims the benefit of Italian patent application 102022000013483 filed 27 Jun. 2022, the disclosures of which are incorporated herein by reference in their entirety.

### TECHNICAL FIELD

The present disclosure relates to a process for recovering and purifying polyhydroxyalkanoates (PHAs) from a fermentation broth.

More particularly, the present disclosure relates to a process for recovering and purifying polyhydroxyalkanoate (PHA) from a fermentation broth deriving from the fermentation of microorganisms comprising the following steps: (a) subjecting the fermentation broth as such to heat treatment in presence of at least one acid; (b) subjecting the fermentation broth obtained in step (a), directly or after storage, to separation obtaining an aqueous suspension of cellular biomass comprising polyhydroxyalkanoate (PHA) and an aqueous phase comprising fermentation residues; (c) subjecting the aqueous suspension of cellular biomass comprising polyhydroxyalkanoate (PHA) obtained in step (b) to extraction, purification and drying; characterized in that said step (a) is carried out at specific values of pH, temperature and time.

The aforementioned polyhydroxyalkanoate (PHA) can be advantageously used in various applications, in particular in the medical, pharmacological, cosmetic, agricultural, engineering and food packaging fields.

### BACKGROUND

Polyhydroxyalkanoates (PHAs) are homopolymers or copolymers of hydroxyalkanoates such as, for example, 3-hydroxybutyrate (3HB), 3-hydroxyvalerate (3HV), 4-hydroxyvalerate (4HV), 3-hydroxyhexanoate (3HH). Said homopolymers or copolymers, obtained from renewable sources, are completely biodegradable and are synthesized and accumulated by various prokaryotic microorganisms, in particular bacteria, as a carbon and energy reserve for cellular metabolism and, in most cases, are accumulated under growing conditions in which an essential nutrient is found in limiting concentrations. The polyhydroxyalkanoates (PHAs) are formed as intracellular granules and can be accumulated up to 80% by weight of the cell mass from which they must then be extracted by operating in such a way as to obtain polyhydroxyalkanoates (PHAs) with a sufficient degree of purity.

Compared to synthetic polymers and other biopolymers obtained from renewable sources [for example, polylactic acid (PLA)], polyhydroxyalkanoates (PHAs) have numerous advantages, particularly in terms of biodegradability, recyclability and hydrophobicity, which make them particularly promising products as biodegradable substitutes for polymers of petrochemical origin.

Furthermore, polyhydroxyalkanoates (PHAs) are of industrial interest as they can be processed with conventional equipment and can, therefore, be exploited in diversified, even with high added value, application fields. The main properties that distinguish them in application, in particular compared to other biopolymers, are the good mechanical and barrier properties (useful, for example, in the field of food packaging) and biocompatibility (useful, for example, in the medical, pharmacological, cosmetic, fields).

The production of polyhydroxyalkanoates (PHAs) is known in the art. However, it should be noted that the production processes of polyhydroxyalkanoates (PHAs) on an industrial scale, with respect to the production processes used for the other biopolymers, are generally more complicated with consequent high production times and costs. In particular, fermentation turns out to be a rather onerous process economically, both due to the cost of the raw materials used and because it is characterized by a rather low final productivity. The recovery and purification of the polyhydroxyalkanoates (PHAs) at the end of the process must therefore be carried out in such a way as to have the maximum possible efficiency without affecting the quality of the produced polyhydroxyalkanoates (PHAs).

As mentioned above, polyhydroxyalkanoates (PHAs) are synthesized by particular prokaryotic microorganisms, in particular bacteria, as an additional source of sustenance to be used in the event that they have to survive in unfavorable conditions (cellular stress). Consequently, the same microorganisms that synthesize and accumulate polyhydroxyalkanoates (PHAs), if they are in unfavorable conditions for growth, are able to activate metabolic processes that lead to the depolymerization of the produced polyhydroxyalkanoates (PHAs). In the processes for the production of polyhydroxyalkanoates (PHAs) by fermentation it is therefore important that said metabolic processes are not triggered in order to maximize the yield of the desired final product.

Both laboratory methods and industrial processes are described in the literature, which can be used to recover and purify the different types of polyhydroxyalkanoates (PHAs) produced by the aforementioned microorganisms by bio-synthetic route. It is also known that the extraction process of the produced polyhydroxyalkanoates (PHAs) has a crucial role in determining their final properties, both in terms of crystallinity and in terms of purity.

For example, Pagliano G. et al., in “Recovery of Polyhydroxyalkanoates From Single and Mixed Microbial Cultures: A Review”, “Frontiers in Bioengineering and Biotechnology” (2021), Vol. 9, Article 624021, report the main parameters that must be analyzed in order to evaluate the production process of polyhydroxyalkanoates (PHA), namely:
recovery efficiency of the obtained polyhydroxyalkanoate (PHA), i.e. the ratio between the polyhydroxyalkanoate (PHA) recovered at the end of the extraction process and that accumulated in the microorganism at the end of the fermentation, measured by laboratory analysis such as, for example, solvent extraction, gas-chromatographic analysis (GC), or thermogravimetric analysis (TGA);
purity of the obtained polyhydroxyalkanoate (PHA), typically determined through thermogravimetric analysis (TGA) (determining, for example, the fixed residue at 600° C.), or through the search for typical contaminants, in particular lipids and proteins;
quality of the obtained polyhydroxyalkanoate (PHA), typically evaluated by determining the molecular weight and polydispersity index of the polyhydroxyalkanoate (PHA) by gel permeation chromatography analysis (GPC).

In the case of intracellular biopolymer production, as is the case of polyhydroxyalkanoates (PHAs), once the synthesis is completed and the fermentation broth has been discharged from the fermentation reactor, said fermentation broth comprising water, fermentation residues (metabolites and organic and/or inorganics compounds used to promote growth) and a cellular biomass comprising polyhydroxyalkanoate (PHA), is subjected to a separation phase carried out using known techniques such as, for example, filtration, centrifugation, flocculation, and subsequent washing through dilution, for example, with demineralized water, obtaining an aqueous suspension of cellular biomass comprising polyhydroxyalkanoate (PHA) and an aqueous phase comprising fermentation residues. Said separation and washing steps can be repeated several times.

The aforementioned aqueous phase is removed as an aqueous stream, while the aforementioned aqueous suspension of cellular biomass comprising polyhydroxyalkanoate (PHA) is subjected to extraction which can be carried out, in the absence of solvent, by cellular, mechanical and/or chemical lysis, during which the cell wall is broken, obtaining an aqueous suspension comprising the organic material deriving from the cell wall breaking, said organic material comprising polypeptides, phospholipids, DNA, RNA, peptidoglycans, in solid form (called Non-PHA Cellular Material—NPCM) and polyhydroxyalkanoate (PHA).

Subsequently, said aqueous suspension comprising the organic material deriving from the breaking of the cell wall (called Non-PHA Cellular Material-NPCM) and polyhydroxyalkanoate (PHA), in order to separate the organic material deriving from the rupture of the cell wall (called Non-PHA Cellular Material—NPCM) from the polyhydroxyalkanoate (PHA), is subjected again to a separation and washing step through dilutions, for example, with demineralized water, as described above, obtaining an aqueous suspension comprising polyhydroxyalkanoate (PHA).

Alternatively, said extraction can be carried out, in the presence of a solvent, by freezing and freeze-drying to obtain a dehydrated cellular powder comprising polyhydroxyalkanoate (PHA) which is subsequently subjected to solubilization in the presence of a solvent.

At the end of the above extraction the purification step is carried out in order to obtain the precipitation of the polyhydroxyalkanoate (PHA), to remove the impurities still present and to increase the purity of the obtained polyhydroxyalkanoate (PHA). Generally, said purification step is carried out through the use of an antisolvent in the case of extraction in the presence of solvent, or, in the case of extraction in the absence of solvent, through one or more washings, for example, with demineralized water, optionally added with an oxidizing agent, to obtain a solid residue comprising purified polyhydroxyalkanoate (PHA).

Subsequently, the obtained solid residue comprising purified polyhydroxyalkanoate (PHA) is subjected to drying in order to remove any antisolvent possibly present and/or water in order to obtain a minimum residual moisture content (i.e. a moisture content lower than or equal to 0.5%) and to allow, therefore, the use of poly hydroxyalkanoate (PHA) in subsequent applications.

It is known in the literature how it is possible and useful to insert an intermediate pre-treatment phase between said separation and washing phase and said extraction phase with the aim of pre-adapting the aqueous suspension of cellular biomass comprising polyhydroxyalkanoate (PHA) to the subsequent extraction phase.

For example, Koller M. et al., in “Strategies for recovery and purification of poly[(R)-3-hydroxyalkanoates] (PHA) biopolyesters from surrounding biomass”, “Engineering in Life Sciences” (2013), Vol. 13, pg. 549-562, report the possibility of drying the aqueous suspension of cellular biomass comprising polyhydroxyalkanoate (PHA) before the cell lysis step by freeze-drying or by thermal treatment (for example, by drying with hot air). The obtained dried cellular biomass comprising polyhydroxyalkanoate (PHA) can be subsequently washed with polar solvents such as, for example methanol, ethanol, acetone, in order to remove the lipids and subjected to the cell lysis step using a solvent (for example, chloroform) obtaining polyhydroxyalkanoate (PHA) with improved purity. The authors also report the possibility of carrying out a mechanical cell lysis, wherein the aqueous suspension of cellular biomass comprising polyhydroxyalkanoate (PHA) is introduced into a homogenizer (2 steps at 800 bar) and of a subsequent chemical cell lysis in the presence of a surfactant (sodium dodecyl sulphate—SDS) (1% with respect to the aqueous suspension of cellular biomass) and a base (sodium hydroxide-NaOH) (pH 12).

Pérez-Rivero C. et al. in “A Sustainable Approach for the Downstream Processing of Bacterial polyhydroxyalkanoates: State-of-the-art and latest developments”, “Biochemical Engineering Journal” (2019), Article 107283, report the pretreatment of the aqueous suspension of cellular biomass comprising polyhydroxyalkanoate (PHA) before the cell lysis step by heating, freezing, adding salts, milling in liquid nitrogen, or using hot compressed water, in order to increase the permeability of the cells of the microorganisms and, consequently, the extraction yield and purity of polyhydroxyalkanoate (PHA).

Kunasundari B. in “Isolation and recovery of microbial polyhydroxyalkanoates”, “eXPRESS Polymer Letters” (2011), Vol. 5, No. 7, pg. 620-634, reports among the pre-treatment techniques of the aqueous suspension of cellular biomass comprising polyhydroxyalkanoate (PHA), the addition of precipitating agents, in particular sodium biphosphate, calcium chloride and polyacrylamide, which allow the cells of the microorganism to be easily separated from rest of the components of the fermentation broth by filtration, for example, through a filter press.

Jiang Y. et al., in “Feasibility study of an alkaline-based chemical treatment for the purification of polyhydroxybutyrate produced by a mixed enriched culture”, “AMB Express” (2015) 5:5, report the efficacy of a cell lysis process effected by addition of a base and a surfactant.

Yu J. et al., in “Generation and Utilization of Microbial Biomass Hydrolysates in Recovery and Production of Poly (3-hydroxybutyrate)”, “IntechOpen—Biomass Now—Cultivation and Utilization” (2013), Chapter 2, pg. 33-48, http://dx.doi.org/10.5772/52940, report a process of extracting poly-3-hydroxybutyrate (PHB) from a fermentation broth comprising subjecting the fermentation broth to centrifugation, acid treatment, basic treatment, preferably in the presence of a surfactant, treatment with hypochlorite and subsequent washing and drying.

The patent U.S. Pat. No. 9,683,076 relates to a process for recovering and purifying polyhydroxyalkanoates (PHA) from cell culture, comprising:
(a) acidifying the cell culture by adding an aqueous solution of an inorganic or organic acid selected from: sulfuric acid, hydrochloric acid, phosphoric acid, nitric acid, acetic acid, citric acid, or mixtures thereof, so as to obtain a pH value less than or equal to 6, and submitting the cell culture to a cell fractionation treatment using high-pressure homogenization at a temperature greater than or equal to 10° C. and lower than or equal to 80° C., so as to obtain a PHA suspension;
(b) basifying the PHA suspension thus obtained so as to obtain a pH value greater than or equal to 8;
(c) diluting the PHA suspension and subjecting the diluted PHA suspension to a first tangential filtration so as to obtain a concentrated PHA suspension as the first retentate and a first aqueous phase as the first permeate;
(d) bleaching the concentrated PHA suspension;
(e) diluting the PHA suspension after bleaching and subjecting the diluted bleached PHA suspension to a second cross-flow filtration to obtain a concentrated bleached PHA suspension as the second retentate and a second aqueous phase as the second permeate; and
(f) subjecting the concentrated bleached PHA suspension to drying.

The above process is said to be advantageous as it is carried out continuously, i.e. in the absence of steps in batches, to separate the PHAs from a suspension and in the absence of solvents and to be able to give PHA in as pure a form as possible without cause a reduction in molecular weight.

The patent EP 1,705,250 relates to a method for directly separating and purifying polyhydroxyalkanoates from cells deriving from bacterial fermentation liquid comprising the following steps:
(1) pretreating the fermentation liquid with physical methods so as to obtain cell membrane break;
(2) adjusting the pH value of the pretreated fermentation liquid to an alkaline value;
(3) adding an anionic surfactant and reacting under agitation;
(4) separating and extracting the precipitate from the reaction liquid;
(5) washing and drying;
   wherein the steps of adjusting the pH and adding a surfactant are interchangeable.

The above process is said to be able to effectively reduce separation and purification costs, to reduce pollution and to be applicable industrially.

The patent U.S. Pat. No. 7,393,668 relates to a method for recovering a polyhydroxyalkanoate from a containing polyhydroxyalkanoate microbial cell comprising the following steps (a) and (b):
(a) a step comprising adding an alkali to an aqueous suspension of the polyhydroxyalkanoate-containing microbial cell while stirring and carrying out a physical disruption treatment to disrupt the cell, solubilizing or emulsifying cell substances other than the polyhydroxyalkanoate in said cell, and then separating the polyhydroxyalkanoate from the aqueous suspension, and
(b) a step comprising treating the separated polyhydroxyalkanoate with an enzyme and/or a surfactant to solubilize impurities adhering to the polyhydroxyalkanoate or to solubilize them after decomposing, and then washing the polyhydroxyalkanoate with a hydrophilic solvent and/or water.

The prior art reported above describes processes for recovering and purifying polyhydroxyalkanoates (PHAs), but does not focus on the development of a process which includes a stabilization step of the fermentation broth as such and its possible coupling with the subsequent extraction and purification steps. In particular, the criticalities that occur when the cells of the microorganism used in the fermentation are in unfavorable environmental conditions are not analyzed, despite being in an active vital state (i.e. absence of substrate and aeration) as typically occurs at the end of fermentation but before cell lysis step. In these conditions, in fact, the cells of the microorganism that are still alive activate metabolic processes which, thanks to the effect of enzymes such as depolymerase, involve the consumption of the polyhydroxyalkanoate (PHA) accumulated inside them during fermentation and which are therefore deleterious, both for the quality and for the amount of the obtainable polyhydroxyalkanoate (PHA). In particular, the depolymerization process of the polyhydroxyalkanoate (PHA) is of the intracellular type and involves the hydrolysis of the polyhydroxyalkanoate (PHA) accumulated inside the cells of the microorganism during fermentation, which is split into simpler elements useful for cellular metabolism as described, for example, by Knoll M. et al., in “The PHA Depolymerase Engineering Database: A systematic analysis tool for the diverse family of polyhydroxyalkanoate (PHA) depolymerases “BMC Bioinformatics” (2009), doi: 10.1186/1471-2105-10-89.

Furthermore, in the prior art reported above, a treatment applicable to the fermentation broth as such is not described which does not impact on the quality of the finished product and which is capable, at the same time, of reducing the losses of polyhydroxyalkanoate (PHA) which are normally generated during the extraction phase due to the residual activity of the cells of the microorganism used or also due to variations in the rheological characteristics of the fermentation broth (for example, the variation in the viscosity of the fermentation broth due to the autolysis phenomenon of the cells of the microorganism). Furthermore, the prior art reported above does not describe how it is appropriate to apply said intermediate pre-treatment step at the very moment in which the conditions favorable to the growth of the cells of the microorganism cease in order to promptly inhibit any metabolic process capable of varying the quality and amount of polyhydroxyalkanoate (PHA) present in the cells of the microorganism, without having to wait for the technical times required for the first operations of treatment of the fermentation broth such as separation and washing. In industrial practice, but also in experimental practice, in cases where it is not possible to treat the fermentation broth instantly, however, it is always advantageous to stabilize said fermentation broth immediately after fermentation and therefore at the very moment in which the favorable conditions to the growth of the cells of the microorganism cease to avoid the onset of the aforementioned metabolic processes that impact on the quality of the final product.

The aforementioned stabilization phase is of particular importance in cases where the first steps of treatment of the fermentation broth consist of laborious or complex operations which require time to be completed especially on a large scale, such as the separation and washing phase which, as mentioned above, can provide for repeated concentrations and dilutions of the fermented broth, and which at an industrial level can require process times of even the order of tens of hours.

A further common problem in the known art is the concentration of the cells of the microorganism in the fermentation broth, on which to carry out the chemical and/or mechanical lysis: normally, in fact, it is necessary to operate at a cell concentration in the fermentation broth lower than 200 g/l (dry weight) as, by increasing the cell concentration, the efficiency and efficacy of the process are reduced.

### SUMMARY

The Applicant has therefore faced the problem of finding a process for the recovery of polyhydroxyalkanoates (PHAs) from a fermentation broth deriving from the fermentation of microorganisms capable of overcoming the aforementioned draw backs.

The Applicant has now found that recovering polyhydroxyalkanoate (PHA) from a fermentation broth deriving from the fermentation of microorganisms, can be advantageously carried out by means of a process comprising the following steps: (a) subjecting the fermentation broth as such to heat treatment in the presence of at least one acid; (b) subjecting the fermentation broth obtained in step (a), directly or after storage, to separation obtaining an aqueous suspension of cellular biomass comprising polyhydroxyalkanoate (PHA) and an aqueous phase comprising fermentation residues; (c) subjecting the aqueous suspension of cellular biomass comprising polyhydroxyalkanoate (PHA) obtained in step (b) to extraction, purification and drying; characterized in that said step (a) is carried out at specific values of pH, temperature and time. Said process allows to inhibit the cellular activity of the cells of the microorganism used in the fermentation and, consequently, the self-consumption of the produced polyhydroxyalkanoate (PHA). In this way, the subsequent steps of separation, extraction, purification and drying, can also be carried out with deferred times, obtaining a yield and quality of the polyhydroxyalkanoate (PHA) in line with those obtainable if the fermentation broth were treated instantaneously. Furthermore, the disclosed process allows for obtaining a variation of the rheological properties of the fermentation broth, which facilitate and make more efficient the polyhydroxyalkanoate (PHA) extraction process, which can be carried out more simply and at higher concentrations than those reported in the known art. Furthermore, the disclosed process can be applied profitably both to extraction processes in the presence of solvent and to extraction processes in the absence of solvent and can therefore also be applied to already existing processes, improving their industrial feasibility. Finally yet importantly, the disclosed process allows the extraction step to be carried out, in the absence of solvent, at high cell concentrations [i.e. at a cell concentration greater than or equal to 200 g/l (dry weight)] maintaining a high efficiency.

The present disclosure therefore provides a process for recovering polyhydroxyalkanoate (PHA) from a fermentation broth deriving from the fermentation of microorganisms comprising the following steps:
(a) subjecting the fermentation broth as such to heat treatment in the presence of at least one acid;
(b) subjecting the fermentation broth obtained in step (a), directly or after storage, to separation obtaining an aqueous suspension of cellular biomass comprising polyhydroxyalkanoate (PHA) and an aqueous phase comprising fermentation residues;
(c) subjecting the aqueous suspension of cellular biomass comprising polyhydroxyalkanoate (PHA) obtained in step (b) to extraction, purification and drying;
   characterized in that said step (a) is carried out at a pH comprised between 4 and 6, preferably comprised between 4.5 and 5.5, more preferably at a pH equal to 5, at a temperature comprised between 50° C. and 70° C., preferably comprised between 55° C. and 65° C., more preferably at a temperature equal to 60° C., for a time comprised between 5 minutes and 30 minutes, preferably comprised between 8 minutes and 20 minutes, more preferably for a time equal to 10 minutes.

For the purpose of the present description and of the following claims, the definitions of the numerical ranges always include the extremes unless otherwise specified.

For the purposes of the present description and of the following claims, the term “comprising” also includes the terms “consisting essentially of” or “consisting of”.

The fermentation broth usable for the purpose of the present disclosure can derive from the fermentation of any microorganism capable of producing polyhydroxyalkanoates (PHAs), in particular of producing intracellular polyhydroxyalkanoates (PHAs).

Specific examples of microorganisms that can be advantageously used for the purpose of the present disclosure belong to the following genera: Cupriavidus, Pseudomonas, Bacillus, Ralstonia, Halomonas, Alcaligenes, Escherichia, Azotobacter, Aeromonas, Nocardia, Methylobaterium, Burkholderia, Hydrogenophaga, preferably the species Cupriavidus necator.

The nutritive substrate can be any type of substrate which can be metabolized by the aforementioned microorganisms in order to produce polyhydroxyalkanoates (PHAs) and can be selected, for example, from: culture media comprising sugars (for example, glucose); juices, molasses or pulps obtained, for example, from the processing of vegetable products such as fruit, sugar beet, sugar cane, oilseeds; wastewater such as, for example, wastewater deriving from water treatment, or from the processing of fruit juices; or similar. Said substrates, in addition to carbohydrates and proteins, generally contain growth factors of various kinds such as, for example, compounds containing nitrogen and/or phosphorus, and other elements useful for the cellular growth of the aforementioned microorganisms.

For the purpose of the present disclosure, said fermentation can be carried out in batch, or in discontinuous culture (fed-batch fermentation), or in continuous culture.

According to a preferred embodiment of the present disclosure, said at least one acid can be selected, for example, from inorganic acids or organic acids such as, for example: sulfuric acid, hydrochloric acid, nitric acid, phosphoric acid, acetic acid, acid citric, preferably sulfuric acid.

At the end of step (a), the obtained fermentation broth is subjected to cooling (for example, by letting the temperature drop spontaneously, or by means of the air- or water-cooling jacket present in the fermentation reactor). According to a preferred embodiment of the present disclosure, the fermentation broth obtained in step (a) can be cooled to a temperature comprised between 5° C. and 40° C., preferably comprised between 10° C. and 30° C., in a time comprised between 5 minutes and 2 hours, preferably comprised between 30minutes and 1 hour.

According to a preferred embodiment of the present disclosure, the fermentation broth obtained in step (a), can be subjected to storage, for a time comprised between 4 hours and 24 hours, preferably comprised between 10 hours and 22 hours.

According to a preferred embodiment of the present disclosure, in said step (b) the fermentation broth obtained in step (a), directly or after storage, can be subjected to tangential filtration and/or centrifugation, which can be carried out either in batch or continuously, more preferably by continuous centrifugation, obtaining an aqueous suspension of cellular biomass comprising polyhydroxyalkanoate (PHA) and an aqueous phase comprising fermentation residues. Preferably, at the end of said filtration and/or centrifugation, a cell concentration comprised between 200 g/l (dry weight) and 800 g/l (dry weight), more preferably comprised between 250 g/l (dry weight) and 500 g/l (dry weight), is obtained.

According to a preferred embodiment of the present disclosure, said aqueous suspension of cellular biomass comprising polyhydroxyalkanoate (PHA), before being subjected to step (c) of extraction, purification and drying, can be subjected to a washing step by dilution with demineralized water or with diluted aqueous solutions containing traces of cellular debris (i.e. diluted aqueous solutions of the aforementioned aqueous phase including fermentation residues) and subsequently, to a concentration phase by tangential filtration and/or centrifugation, which can be carried out in batch or continuously, preferably by continuous centrifugation. Preferably, at the end of said washing step, a cell concentration comprised between 100 g/l (dry weight) and 400 g/l (dry weight), preferably comprised between 150 g/l (dry weight) and 250 g/l is obtained. 1 (dry weight), while at the end of said concentration step a cell concentration comprised between 200 g/l (dry weight) and 800 g/l (dry weight), preferably comprised between 250 g/l (dry weight) and 500 g/l (dry weight) is obtained.

The above washing and concentration steps can possibly be repeated several times.

The cellular concentration of the aqueous suspension of cellular biomass comprising polyhydroxyalkanoate (PHA) obtained can be measured in grams per liter of broth, by determining the dry weight of the cells of the microorganism used from a broth sample of known volume taken at pre-set intervals such as, for example, during and/or at the end of the fermentation, and/or at the end of said washing and concentration steps. In particular, by dry weight of the cellular biomass we mean the weight of the cells of the microorganism contained in a known volume of broth, determined by weighing the aforementioned cells of the microorganism after having eliminated all the water content by filtration on Whatman GF filters/F (0.7 μm) and subsequent heat treatment in a ventilated oven at 105° C. until constant weight (about 24 hours).

The aqueous suspension of cellular biomass comprising polyhydroxyalkanoate (PHA) obtained in step (b) of separation is subjected to step (c) of extraction, purification and drying.

Step (c) of extraction, purification and drying of the process of the present disclosure can be carried out in the presence or absence of a solvent.

According to a preferred embodiment of the present disclosure, in the event that said step (c) is carried out in the absence of solvent, the aqueous suspension of cellular biomass comprising polyhydroxyalkanoate (PHA) obtained in (b) of separation can be subjected to mechanical and/or chemical cell lysis.

According to a preferred embodiment of the present disclosure, said aqueous suspension of cellular biomass comprising polyhydroxyalkanoate (PHA), can be subjected to cell lysis by mechanical treatment, which can be carried out using high pressure homogenizers (for example, homogenizer Mod. PandaPLUS 2000of the Gea), at a pressure comprised between 500 bar and 2000 bar, preferably comprised between 800 bar and 1500 bar, at a temperature comprised between 10° C. and 80° C., preferably comprised between 20° C. and 50° C. Generally, said mechanical treatment requires from one to five steps in a homogenizer and is carried out at a temperature and pressure such as to avoid possible degradation of the poly hydroxyalkanoate (PHA).

According to a preferred embodiment of the present disclosure, at the end of said mechanical treatment, the aqueous suspension comprising polyhydroxyalkanoate (PHA) obtained can be subjected to chemical cell lysis, said chemical cell lysis comprising:
basifying said aqueous suspension comprising polyhydroxyalkanoate (PHA) until obtaining a pH value greater than or equal to 9, preferably comprised between 9.5 and 10, by adding at least one strong base, preferably an aqueous solution of at least one inorganic strong base such as, for example, sodium hydroxide, potassium hydroxide, or mixtures thereof, obtaining a basified aqueous suspension comprising polyhydroxyalkanoate (PHA);
adding to said basified aqueous suspension comprising polyhydroxyalkanoate (PHA), at least one surfactant in an amount comprised between 0.05 to 1 g, preferably comprised between 0.1 to 0.5 g, per gram of dry weight of said aqueous suspension comprising polyhydroxyalkanoate (PHA) based.

According to a preferred embodiment of the present disclosure, said chemical cell lysis can be carried out at a temperature comprised between 10° C. and 80° C., preferably comprised between 20° C. and 50° C., for a time comprised between 20 minutes and 120 minutes, preferably comprised between 50 minutes and 70 minutes.

Said surfactant can be selected, for example, from anionic, cationic or non-ionic surfactants. Said surfactants are preferably selected from those with a low environmental impact, so as to avoid disposal problems.

Anionic surfactants useful for the purpose of the present disclosure can be selected, for example, from alkyl or alkenyl sulphates, alkyl or alkenyl benzene sulphonates, alkyl or alkenyl ether sulphates, alkyl or alkenyl carboxylates, alkyl or alkenyl ether carboxylates, or mixtures thereof. Particularly preferred are the C₁₀-C₁₈ alkyl sulphates. more preferred is sodium dodecyl sulphate (SDS).

Cationic surfactants suitable for the purpose of the present disclosure can be selected, for example, from alkyltrimethylammonium or dialkyldimethylammonium salts, or mixtures thereof.

Non-ionic surfactants suitable for the purpose of the present disclosure can be selected, for example, from polyoxyalkylenes (preferably polyoxyethylene) alkyl or alkenyl ethers, alkyl or alkenyl phenyl ethers, polyoxyethylene/poly oxypropylene copolymers, or mixtures thereof.

According to a preferred embodiment of the present disclosure, the aqueous suspension comprising polyhydroxyalkanoate (PHA) obtained from chemical cell lysis can be subjected to a first step of concentration by tangential filtration and/or centrifugation, which can be carried out in batch or continuously, preferably by continuous centrifugation, subsequently subjected to a washing phase by dilution with demineralized water or with diluted aqueous solutions containing traces of cellular debris, and subsequently subjected to a second concentration step by tangential filtration and/or centrifugation, which can be carried out in batch or continuously, preferably by continuous centrifugation. Preferably, at the end of said first concentration step, a cell concentration comprised between 200 g/l (dry weight) and 800 g/l (dry weight), more preferably comprised between 250 g/l (dry weight) and 500 g/l (dry weight) is obtained, while at the end of said washing step, a cell concentration comprised between 100 g/l (dry weight) and 400 g/l, more preferably comprised between 150 g/l (dry weight) and 200 g/l (dry weight), and at the end of said second concentration step a cell concentration comprised between 200 g/l (dry weight) and 800 g/l (dry weight), preferably comprised between 250 g/l (dry weight) and 500 g/l (dry weight) is obtained.

The above concentration and dilution steps can possibly be repeated several times.

It should be noted that after mechanical and/or chemical cell lysis the term “cell concentration” refers to the concentration of the organic material deriving from the breaking of the cell wall, said organic material comprising polypeptides, phospholipids, DNA, RNA, peptidoglycans, in the form solid (called Non-PHA Cellular Material—NPCM) and polyhydroxyalkanoate (PHA). In particular, dry weight means the weight of said organic material and said polyhydroxyalkanoate (PHA) contained in a known volume of aqueous suspension comprising polyhydroxyalkanoate (PHA), determined by weighing said organic material and polyhydroxyalkanoate (PHA) after all the water content was eliminated by filtration on Whatman GF/F filters (0.7 μm) and subsequent heat treatment in a ventilated oven at 105° C. until constant weight (about 24 hours).

According to a preferred embodiment of the present disclosure, the aqueous suspension comprising polyhydroxyalkanoate (PHA) obtained from chemical cell lysis can be subjected to drying, preferably in a ventilated oven, at a temperature greater than or equal to 50° C., preferably comprised between 55° C. and 70° C., until a solid residue comprising polyhydroxyalkanoate (PHA) having a residual humidity lower than 0.5% is obtained.

According to a preferred embodiment of the present disclosure, in the event that said step (c) is carried out in the presence of a solvent, the aqueous suspension of cellular biomass comprising polyhydroxyalkanoate (PHA) obtained in step (b) of separation can be subjected to freezing and freeze-drying.

According to a preferred embodiment of the present disclosure, the aqueous suspension of cellular biomass comprising polyhydroxyalkanoate (PHA) obtained in step (b) can be subjected to a process comprising:
subjecting said aqueous suspension of cellular biomass comprising polyhydroxyalkanoate (PHA) to freezing, at −20° C., and subsequent freeze-drying carried out at −46° C., 0.15 mbar, for a time greater than or equal to 12 hours, preferably comprised between 18 hours and 24 hours, until a dehydrated cellular powder comprising polyhydroxyalkanoate (PHA) is obtained;
subjecting the dehydrated cellular powder comprising polyhydroxyalkanoate (PHA) to washing with ethanol, at a temperature comprised between 30° C. and 60° C., preferably 50° C., for a time comprised between 30 minutes and 6 hours, preferably 4 hours, by working at a dehydrated cellular powder: ethanol ratio comprised between 1:10 to 1:40, preferably at a ratio of 1:20, obtaining a suspension comprising polyhydroxyalkanoate (PHA);
filtering the obtained suspension comprising polyhydroxyalkanoate (PHA) with a porous septum filter or a cellulose filter and subjecting the solid residue comprising polyhydroxyalkanoate (PHA) left on the filter to drying, at room temperature (25° C.), for a time greater than or equal to 12 hours, preferably comprised between 18 hours and 24 hours;
subjecting the obtained dried solid residue comprising polyhydroxyalkanoate (PHA) to solubilization in the presence of at least one solvent which can be selected, for example, from chloroform, 1,2-dichloroethane, dichloromethane, preferably chloroform, in a stirred vessel or in a reflux reactor, operating at a dry residue:solvent ratio comprised between 1:100 and 1:500, preferably equal to 1:200, for a time comprised between 4 hours and 24 hours, preferably 8 hours, at a temperature comprised between 40° C. and 70° C., preferably 60° C. and, optionally, repeating said solubilization up to 3 times;
subjecting the solution comprising polyhydroxyalkanoate (PHA) obtained after solubilization to filtration with a septum filter or with a cellulose filter obtaining a solid residue free of polyhydroxyalkanoate (PHA) and a solution comprising polyhydroxyalkanoate (PHA);
optionally, concentrating the solution comprising polyhydroxyalkanoate (PHA) obtained by operating at a temperature lower than or equal to 70° C., preferably comprised between 40° C. and 60° C., at a pressure lower than atmospheric pressure, preferably equal to 0.5 bar;
placing the solution comprising polyhydroxyalkanoate (PHA), before or after possible concentration, in contact with an excess of ethanol cooled to −20° C., in a stirred vessel, for a time comprised between 0.1 minutes and 60 minutes, preferably comprised between 0.5 minutes and 25 minutes, obtaining the precipitation of a solid residue comprising polyhydroxyalkanoate (PHA);
mechanically collecting the obtained solid residue comprising polyhydroxyalkanoate (PHA) and subjecting it to drying at room temperature (25° C.), for a time greater than or equal to 12 hours, preferably comprised between 20 hours and 30 hours, or, in a ventilated oven, at a temperature equal to 50° C., at low pressure (for example, 11 absolute mbar), until a solid residue comprising polyhydroxyalkanoate (PHA) having a residual humidity lower than 0.5% is obtained.

As regards the polyhydroxyalkanoates (PHAs) which can be produced according to the present disclosure, these are generally polymers containing repeating units having the general formula (I):

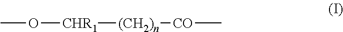

wherein R₁ represents a hydrogen atom, or is selected from C₁-C₁₂ alkyl groups. C₄-C₁₆ cycloalkyl groups, C₂-C₁₂ alkenyl groups, optionally substituted with at least one group selected from halogens such as, for example, fluorine, chlorine, bromine, —CN, —OH, —COOH, —OR₃, —COOR₃ wherein R₃ represents a C₁-C₄ alkyl group or a benzyl group, n is an integer comprised between 1 and 6, preferably 1 or 2. Preferably Ri is methyl or ethyl and n is 1 or 2.

Polyhydroxyalkanoates (PHAs) can be both homopolymers and copolymers or terpolymers. In the case of copolymers or terpolymers, they can consist of different repeating units having general formula (I) in combination with at least one repeating unit deriving from comonomers which are capable of copolymerizing with hydroxyalkanoates, for example lactones or lactams. In the latter case, the repeating units having general formula (I) are present in an amount equal to at least 10% by moles with respect to the total moles of repeating units.

Repeating units having general formula (I) are those deriving from: 3-hydroxybutyrate, 3-hydroxyvalerate, 3-hydroxyhexanoate, 3-hydroxyoctanoate, 3-hydroxyundec-10-enoate, 4-hydroxyvalerate.

Particularly preferred polyhydroxyalkanoates (PHAs) are: poly-3-hydroxybutyrate (PHB), poly-3-hydroxyvalerate (PHV), poly-3-hydroxyhexanoate (PHH), poly-3-hydroxyoctanoate (PHO), poly (3-hydroxybutyrate-co-3-hydroxy-valerate) (PHBV), poly(3-hydroxybutyrate-co-3-hydroxyhexanoate) (PHBH), poly(3-hydroxybutyrate-co-4-hydroxybutyrate) (PHBB), poly(3-hydroxyoctanoate-co-3-hydroxy-undecen-10-enoate) (PHOU), poly (3-hydroxybutyrate-co-3-hydroxyvalerate-c-4-hydroxyvalerate) (PHBVV), or mixtures thereof. Poly-3-hydroxybutyrate (PHB) is particularly preferred.

### BRIEF DESCRIPTION OF THE DRAWINGS

FIG. 1 and FIG. 2 shown below show two embodiments of the disclosed process provided for the sole purpose of illustration and not for limitation of the same.

### DETAILED DESCRIPTION OF THE DRAWINGS AND THE DISCLOSURE

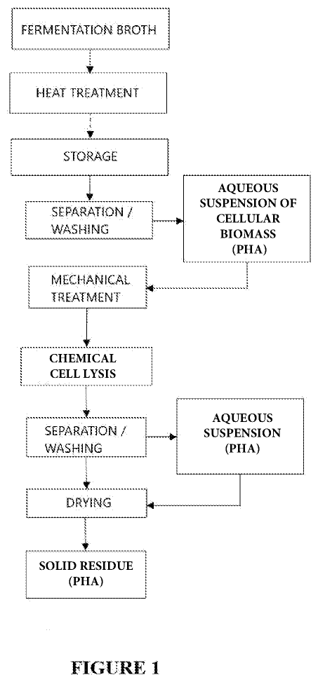

In particular, FIG. 1 illustrates an embodiment provided of the present disclosure wherein the step (c) of extraction, purification and drying is carried out in the absence of solvent. For this purpose, the fermentation broth is subjected to heat treatment, in the presence of at least one acid (for example, sulfuric acid) [step (a)]. Subsequently, the fermentation broth obtained in step (a) is subjected, directly or after storage (indicated with a dashed line in FIG. 1), to separation (for example, by continuous centrifugation) and subsequent washing (for example, by dilution with distilled water) (said separation/washing phase can possibly be repeated several times) obtaining an aqueous suspension of cellular biomass comprising polyhydroxyalkanoate (PHA) and an aqueous phase comprising fermentation residues (not represented in FIG. 1). The aqueous suspension of cellular biomass comprising polyhydroxyalkanoate (PHA) obtained is subjected to mechanical treatment (i.e. mechanical cell lysis) (for example, in a homogenizer) and, subsequently, to chemical cell lysis (for example, in the presence of at least one base and of at least one surfactant). The aqueous suspension comprising polyhydroxyalkanoate (PHA) obtained at the end of the cellular chemical lysis (not represented in FIG. 1) is subjected to separation (for example, by continuous centrifugation) and subsequent washing (for example, by dilution with distilled water) (said separation/washing step can optionally be repeated several times) obtaining an aqueous suspension comprising polyhydroxyalkanoate (PHA) which is subjected to drying (for example, in a ventilated oven) obtaining a solid residue comprising polyhydroxyalkanoate (PHA).

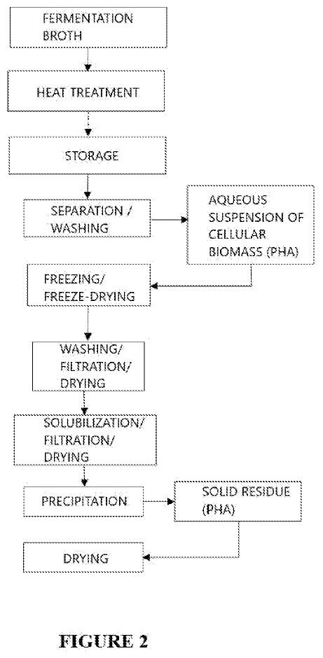

FIG. 2 illustrates an embodiment provided of the present disclosure wherein step (c) of extraction, purification and drying is carried out in the presence of a solvent. For this purpose, the fermentation broth is subjected to heat treatment, in the presence of at least one acid (for example, sulfuric acid) [step (a)]. Subsequently, the fermentation broth obtained in step (a) is subjected, directly or after storage (indicated with a dashed line in FIG. 2), to separation (for example, by continuous centrifugation) and subsequent washing (for example, by dilution with distilled water) (said separation/washing phase can possibly be repeated several times) obtaining an aqueous suspension of cellular biomass comprising polyhydroxyalkanoate (PHA) and an aqueous phase comprising fermentation residues (not represented in FIG. 2). The aqueous suspension of obtained cellular biomass comprising polyhydroxyalkanoate (PHA) is subjected to freezing/freeze-drying obtaining a dehydrated cellular powder comprising polyhydroxyalkanoate (PHA) (not represented in FIG. 2) which is subjected to washing (for example, with ethanol) obtaining a suspension comprising polyhydroxyalkanoate (PHA) (not represented in FIG. 2) which is subjected to filtration obtaining a solid residue comprising polyhydroxyalkanoate (PHA) (not represented in FIG. 2) which is subjected to drying (for example, at room temperature). The solid residue comprising dried polyhydroxyalkanoate (PHA) is solubilized in the presence of at least one solvent (for example, chloroform) and the obtained solution comprising polyhydroxyalkanoate (PHA) (not represented in FIG. 2) was filtered obtaining a solid residue free of polyhydroxyalkanoate (PHA) (not represented in FIG. 2) and a solution comprising polyhydroxyalkanoate (PHA) (not represented in FIG. 2). The solution comprising polyhydroxyalkanoate (PHA) is precipitated in the presence of an excess of cold ethanol obtaining a solid residue comprising polyhydroxyalkanoate (PHA) which is subjected to drying (for example, at room temperature or in a ventilated oven).

In order to better understand the present disclosure and to put it into practice, some illustrative and non-limiting examples of the same are given below.

### Example 1 (Disclosure) [Step (c) in the Absence of Solvent]

For this purpose, a fermentation was carried out with cells of Cupravidus necator DSM 545 starting from a substrate of glucose and salts [(NH₄)₂SO₄, MgSO₄, KH₂PO₄, Na₂HPO₄], citric acid and metals in traces, obtaining a cell concentration equal to 150 g/l (dry weight) containing 70% by weight of poly-3-hydroxybutyrate (PHB).

The poly-3-hydroxybutyrate (PHB) content was determined by thermogravimetric analysis (TGA) according to the UNI EN ISO 11358-1:2014 standard.

2 liters of the obtained fermentation broth were placed in a 5 liter beaker and, after adding a 10% sulfuric acid aqueous solution, previously prepared from 95%-97% sulfuric acid (EMSURE® ISO-Merck), in an amount such as to bring the pH to a value equal to 5, the whole was heated to 60° C. and kept for 10 minutes, at said temperature, under stirring (250 rpm). Subsequently, the fermentation broth was cooled by letting the temperature drop spontaneously to room temperature (25° C.) and subjected to centrifugation in a fixed rotor laboratory centrifuge (6000 rpm, 10 minutes) and the obtained aqueous suspension of cellular biomass (pellet) was diluted to the initial cell concentration (i.e. 150 g/l) (dry weight) by adding demineralized water.

Subsequently, the diluted aqueous suspension of cellular biomass (pellet) was subjected again to centrifugation in a fixed rotor laboratory centrifuge (6000 rpm, 10 minutes) and the obtained aqueous suspension of concentrated cellular biomass (pellet) was diluted by adding demineralized water at a cell concentration of 250 g/l (dry weight).

The aqueous suspension of the obtained diluted cellular biomass (pellet) was subjected to mechanical lysis through a passage in a homogenizer (Mod. PandaPLUS 2000 by Gea), at 1000 bar, at room temperature (25° C.) and subsequently diluted by adding demineralized water at a cell concentration of 100 g/l (dry weight). The aqueous suspension thus obtained was subjected to chemical lysis by adding a 20% sodium hydroxide aqueous solution, previously prepared from sodium hydroxide (purity ≥ 98%—Merck) in an amount such as to bring the pH to a value equal to 9.6 and 0.15 g of sodium dodecyl sulphate (SDS) (purity 99%—Thermo Fisher Scientic) per g of dry weight: the whole was kept, under stirring (250 rpm), for 1 hour, at room temperature (25° C.).

Subsequently, the whole was centrifuged using a fixed rotor laboratory centrifuge (6000 rpm, 20 minutes) and the obtained aqueous suspension (pellet) was diluted by adding demineralized water to the initial cell concentration (i.e.

100 g/l) (dry weight) and centrifuged again using a fixed rotor laboratory centrifuge (6000 rpm, 20 minutes). The obtained concentrated aqueous suspension (pellet) was dried in a ventilated oven, at 65° C., for 12 hours, obtaining a solid residue comprising poly-3-hydroxybutyrate (PHB).

The obtained solid residue comprising poly-3-hydroxybutyrate (PHB) was subjected to thermogravimetric analysis (TGA) according to the UNI EN ISO 11358-1:2014TGA standard, obtaining a solid residue, at 600° C., equal to 0.4% [solid residue not including poly-3-hydroxybutyrate—(PHB)].

The determination of the weight average molecular weight (M_{w}) of the obtained poly-3-hydroxybutyrate (PHB) was carried out by subjecting the solid residue comprising poly-3-hydroxybutyrate (PHB) to GPC (Gel Permeation Chromatography), using UltiMate 3000 Dionex by Thermo Fischer Scientific using refractive index (RI) and operating under the following conditions:
column TSKgel GMHHr-M (300×7,8 mm) with pre-column;
solvent/eluent: chloroform;
flow: 0.5 ml/min;
temperature: 35° C.;
Polystyrene (PS) Standard Kit [10 PS polymers with peak molecular weight (Mₚ) from 580 g/ml to 6570000 g/mol];
   and it turned out to be equal to 1.6×10⁶ Daltons.

The extraction yield of the whole process expressed as follows: grams of poly-3-hydroxybutyrate (PHB) obtained at the end of the process/grams of poly-3-hydroxybutyrate (PHB) synthesized from the microorganism used and available at the time of discharging from the fermenter was found to be >98%.

### Example 2 (Disclosure) [Step (c) in the Presence of Solvent]

For this purpose, a fermentation was carried out with cells of Cupravidus necator DSM 545 starting from a substrate of glucose and salts [(NH₄)₂SO₄, MgSO₄, KH₂PO₄, Na₂HPO₄], citric acid and metals in traces, obtaining a concentration of cellular biomass equal to 150 g/l (dry weight) containing 70% by weight of poly-3-hydroxybutyrate (PHB).

The poly-3-hydroxybutyrate (PHB) content was determined by thermogravimetric analysis (TGA) according to the UNI EN ISO 11358-1:2014standard.

2 liters of the obtained fermentation broth were placed in a 5 liter beaker and, after adding a 10% sulfuric acid aqueous solution, previously prepared from 95%-97% sulfuric acid (EMSURE® ISO-Merck), in an amount such as to bring the pH to a value equal to 5, the whole was heated to 60° C. and kept for 10 minutes, at said temperature, under stirring (250 rpm). Subsequently, the fermentation broth was cooled by letting the temperature drop spontaneously to room temperature (25° C.) and subjected to centrifugation in a fixed rotor laboratory centrifuge (6000 rpm, 10 minutes) and the obtained aqueous suspension of cellular biomass (pellet) was diluted to the initial cell concentration (ie 150 g/l) (dry weight) by adding demineralized water and again subjected to centrifugation in a fixed rotor laboratory centrifuge (6000 rpm, 10 minutes).

Subsequently, the obtained aqueous suspension of concentrated cellular biomass (pellet) was frozen at −20° C. and dried in a freeze dryer at −46° C., 0.15 mbar, for 24 hours. The obtained dehydrated cellular powder was washed with ethanol (96°—Carlo Erba) (dehydrated cellular powder:ethanol ratio 1:20), at a temperature of 50° C., for 4 hours: subsequently, filtration was carried out through porous septum filter obtaining a solid residue comprising poly-3-hydroxybutyrate (PHB) which remains on the filter. The obtained solid residue was left to dry at room temperature (25° C.) for 24 hours.

Subsequently, the dried solid residue was subjected to solubilization in chloroform (purity ≥99.8%—HiPerSolv CHROMANORM®—VWR) (ratio dried solid residue:chloroform equal to 1:200), at a temperature equal to 60° C., for 8hours: said solubilization was repeated 3 times.

Subsequently, the whole was subjected to filtration through a porous septum filter obtaining a solid residue remaining on the filter, not comprising poly-3-hydroxybutyrate (PHB) and a solution comprising poly-3-hydroxybutyrate (PHB), which was placed in contact with an excess of ethanol (96°—Carlo Erba), at −20° C., for 10 minutes: the obtained precipitate comprising poly-3-hydroxybutyrate (PHB) was dried, at room temperature (25° C.), for 12 hours, giving a solid residue comprising poly-3-hydroxybutyrate (PHB).

The obtained solid residue comprising poly-3-hydroxybutyrate (PHB) was subjected to thermogravimetric analysis (TGA) according to the UNI EN ISO 11358-1:2014 TGA standard obtaining a solid residue, at 600° C., equal to 0.1% [solid residue not including poly-3-hydroxybutyrate-(PHB)].

The determination of the weight average molecular weight (M_{w}) of the obtained poly-3-hydroxybutyrate (PHB) was carried out by GPC (Gel Permeation Chromatography), operating as described above in Example 1 and it resulted to be equal to 1, 6×10⁶ Daltons.

The extraction yield of the whole process expressed as follows: grams of poly-3-hydroxybutyrate (PHB) obtained at the end of the process/grams of poly-3-hydroxybutyrate (PHB) synthesized from the microorganism used and available at the time of discharging from the fermenter was found to be >99%.

### Example 3 (Comparative [Absence of Step (a) of Treatment of the Fermentation Broth])

For this purpose, a fermentation was carried out with cells of Cupravidus necator DSM 545 starting from a substrate of glucose and salts [(NH₄)₂SO₄, MgSO₄, KH₂PO₄, Na₂HPO₄], citric acid and metals in traces, obtaining a concentration of cellular biomass equal to 150 g/l (dry weight) containing 70% of poly-3-hydroxybutyrate (PHB).

The poly-3-hydroxybutyrate (PHB) content was determined by thermogravimetric analysis (TGA) according to the UNI EN ISO 11358-1:2014 standard.

2 liters of the fermentation broth, 5 hours after the end of fermentation in which it was kept at room temperature (25° C.), was subjected to centrifugation in a fixed rotor laboratory centrifuge (6000 rpm, 10 minutes): in this case, it has not been possible to obtain a clear separation of the solid phase from the liquid phase, not even by increasing centrifugation speeds and times.

Given the results, it was decided not to proceed with the extraction procedure and to evaluate the content of poly-3-hydroxybutyrate (PHB) in the cells present in the partially separated solid phase by thermogravimetric analysis (TGA) according to the UNI EN ISO 11358-1:2014 standard.

Evaluating the difference in the results of the two thermogravimetric analyzes (TGA) carried out, the first on the cellular broth as it is immediately after discharging from the fermenter and the second on the cellular material recovered after centrifugation carried out starting 5 hours after fermentation, it was possible to estimate that about 10% by weight of the poly-3-hydroxybutyrate (PHB) originally contained in the cells was no longer available.

### Example 4 (Disclosure) [Step (c) in the Presence of Solvent-Extraction at 20 Hours]

For this purpose, a fermentation was carried out with cells of Cupravidus necator DSM 545 starting from a substrate of glucose and salts [(NH₄)₂SO₄, MgSO₄, KH₂PO₄, Na₂HPO₄], citric acid and metals in traces, obtaining a concentration of cellular biomass equal to 150 g/l (dry weight) containing 70% by weight of poly-3-hydroxybutyrate (PHB).

The content of poly-3-hydroxybutyrate (PHB) was determined by thermogravimetric analysis (TGA) according to the UNI EN ISO 11358-1:2014 standard.

2 liters of the obtained fermentation broth were placed in a 5 liter beaker and, after the addition of a 10% sulfuric acid aqueous solution, previously prepared from 95%-97% sulfuric acid (EMSURE® ISO-Merck), in an amount such as to bring the pH to a value equal to 5, the whole was heated to 60° C. and kept, at said temperature, under stirring (250 rpm), for 10 minutes. Subsequently, the fermentation broth was cooled allowing the temperature to rise spontaneously to room temperature (25° C.). After 20 hours of storage at room temperature (25° C.), the fermentation broth was subjected to centrifugation in a fixed rotor laboratory centrifuge (6000 rpm, 10 minutes) and the obtained aqueous suspension of concentrated cellular biomass (pellet) was diluted to the initial cell concentration (i.e. 150 g/l) (dry weight) by adding demineralized water and subjected to centrifugation again in a fixed rotor laboratory centrifuge (6000 rpm, 10 minutes).

Subsequently, the obtained aqueous suspension of concentrated cellular biomass (pellet) was frozen at −20° C. and dried in a freeze dryer at −46° C., 0.15 mbar, for 24 hours. The obtained dehydrated cellular powder was washed with ethanol (96°—Carlo Erba) (dehydrated cellular powder:ethanol ratio 1:20), at a temperature of 50° C., for 4 hours: subsequently, filtration was carried out through porous septum filter obtaining a solid residue comprising poly-3-hydroxybutyrate (PHB) which remains on the filter. The obtained solid residue was left to dry at room temperature (25° C.) for 24 hours.

Subsequently, the dried solid residue was subjected to solubilization in chloroform (purity ≥ 99.8%-HiPerSolv CHROMANORM &-VWR) (dried solid residue:chloroform ratio equal to 1:200), at a temperature equal to 60° C., for 8 hours: said solubilization was repeated 3 times.

Subsequently, the whole was subjected to filtration through a porous septum filter obtaining a solid residue remaining on the filter not comprising poly-3-hydroxybutyrate (PHB) and a solution comprising poly-3-hydroxybutyrate (PHB) which was placed in contact with an excess of ethanol (96°—Carlo Erba), at −20° C., for 10 minutes: the obtained precipitate comprising poly-3-hydroxybutyrate (PHB) was dried, at room temperature (25° C.), for 12 hours, giving a solid residue comprising poly-3-hydroxybutyrate (PHB).

The obtained solid residue comprising poly-3-hydroxybutyrate (PHB) was subjected to thermogravimetric analysis (TGA) according to the UNI EN ISO 11358-1:2014 TGA standard obtaining a solid residue, at 600° C., equal to 0.1% by weight [solid residue not including poly-3-hydroxybutyrate—(PHB)].

The determination of the weight average molecular weight (M_{w}) of the obtained poly-3-hydroxybutyrate (PHB) was carried out by GPC (Gel Permeation Chromatography), operating as described above in Example 1 and it resulted to be equal to 1,6×10⁶ Daltons.

The extraction yield of the whole process expressed as follows: grams of poly-3-hydroxybutyrate (PHB) obtained at the end of the process/grams of poly-3-hydroxybutyrate (PHB) synthesized from the microorganism used and available at the time of discharging from the fermenter was found to be >99%.

### Example 5 (Disclosure) [Step (c) in the Absence of Solvent-High Cell Concentration]

For this purpose, a fermentation was carried out with cells of Cupravidus necator DSM 545 starting from a substrate of glucose and salts [(NH₄)₂SO₄, MgSO₄, KH₂PO₄, Na₂HPO₄], citric acid and metals in traces, obtaining a concentration of cellular biomass equal to 150 g/l (dry weight) containing 70% of poly-3-hydroxybutyrate (PHB).

The poly-3-hydroxybutyrate (PHB) determined by thermogravimetric analysis (TGA) according to the UNI EN ISO 11358-1:2014 standard.

2 liters of the obtained fermentation broth were placed in a 5 liter beaker and, after adding a 10% sulfuric acid aqueous solution, previously prepared from 95%-97% sulfuric acid (EMSURE® ISO—Merck), in an amount such as to bring the pH to a value equal to 5, the whole was heated to 60° C. and kept, at said temperature, under stirring (250 rpm), for 10 minutes. Subsequently, the fermentation broth was cooled by letting the temperature drop spontaneously to room temperature (25° C.) and subjected to centrifugation in a fixed rotor laboratory centrifuge (6000 rpm, 10 minutes) and the obtained aqueous suspension of cellular biomass (pellet) was diluted to the initial cell concentration (i.e. 150 g/l) (dry weight) by adding demineralized water.

Subsequently, the obtained aqueous suspension of diluted cellular biomass (pellet) was subjected again to centrifugation in a fixed rotor laboratory centrifuge (6000 rpm, 10 minutes) and the obtained aqueous suspension of concentrated cellular biomass (pellet) was diluted by adding of demineralized water at a cell concentration of 250 g/l (dry weight).

The obtained aqueous suspension of diluted cellular biomass (pellet) was subjected to mechanical lysis through a passage in a homogenizer (Mod. PandaPLUS 2000 by Gea), at 1000 bar, at room temperature (25° C.) and then diluted with demineralized water at a cell concentration of 200 g/l (dry weight). The obtained aqueous suspension thus was subjected to chemical lysis by adding a 20% sodium hydroxide aqueous solution, previously prepared from sodium hydroxide (purity ≥98%—Merck) in such an amount as to bring the pH to a value equal to 9.6 and 0.15 g of sodium dodecyl sulphate (SDS) (purity 99%—Thermo Fisher Scientic) per g of dry weight: the whole was kept under stirring for 1 hour at room temperature (25° C.).

Subsequently, the whole was centrifuged by fixed rotor laboratory centrifuge (6000 rpm, 20 minutes) and the obtained concentrated aqueous suspension (pellet) was diluted to the initial cell concentration (i.e. 200 g/l) (dry weight) by addition of demineralized water and centrifuged again using a fixed rotor laboratory centrifuge (6000 rpm, 20 minutes). The obtained concentrated aqueous suspension (pellet) was dried in a ventilated oven, at 65° C., for 12 hours, obtaining a solid residue comprising poly-3-hydroxybutyrate (PHB).

The obtained solid residue comprising poly-3-hydroxybutyrate (PHB) was characterized by thermogravimetric analysis (TGA) according to UNI EN ISO 11358-1:2014 TGA obtaining a solid residue, at 600° C., equal to 0.4% by weight [solid residue not including poly-3-hydroxybutyrate—(PHB)].

The determination of the weight average molecular weight (M_{w}) of the obtained poly-3-hydroxybutyrate (PHB) was carried out by GPC (Gel Permeation Chromatography), operating as described above in Example 1 and it resulted to be equal to 1,6×10⁶ Daltons.

The extraction yield of the whole process expressed as follows: grams of poly-3-hydroxybutyrate (PHB) obtained at the end of the process/grams of poly-3-hydroxybutyrate (PHB) synthesized from the microorganism used and available at the time of discharging from the fermenter was found to be >84%.

### Example 6 (Comparative [Absence of Step (a) of Treatment of the Fermentation Broth])

For this purpose, a fermentation was carried out with cells of Cupravidus necator DSM 545 starting from a substrate of glucose and salts [(NH₄)₂SO₄, MgSO₄, KH₂PO₄, Na₂HPO₄], citric acid and metals in traces, obtaining a concentration of cellular biomass equal to 150 g/l (dry weight) containing 70% by weight of poly-3-hydroxybutyrate (PHB).

The poly-3-hydroxybutyrate (PHB) content was determined by thermogravimetric analysis (TGA) according to the UNI EN ISO 11358-1:2014 standard.

2 liters of the obtained fermentation broth were placed in a 5 liter beaker and, after adding a 10% sulfuric acid aqueous solution, previously prepared from 95%-97% sulfuric acid (EMSURE® ISO—Merck), in an amount such as to bring the pH to a value equal to 5, the whole was heated to 60° C. and kept for 10 minutes, at said temperature, under stirring (250 rpm). Subsequently, the fermentation broth was cooled by letting the temperature drop spontaneously to room temperature (25° C.) and subjected to centrifugation in a fixed rotor laboratory centrifuge (6000 rpm, 10 minutes) and the obtained aqueous suspension of cellular biomass (pellet) was diluted to the initial cell concentration (i.e. 150 g/l) (dry weight) by adding demineralized water and again subjected to centrifugation in a fixed rotor laboratory centrifuge (6000 rpm, 10 minutes).

Subsequently, the obtained aqueous suspension of concentrated cellular biomass (pellet) was frozen at −20° C. and dried in a freeze dryer at −46° C., 0.15 mbar, for 24 hours. The obtained dehydrated cellular powder was washed with ethanol (96°-Carlo Erba) (dehydrated cellular powder:ethanol ratio 1:20), at a temperature of 50° C., for 4 hours: subsequently, filtration was carried out through porous septum filter obtaining a solid residue comprising poly-3-hydroxybutyrate (PHB) which remains on the filter. The obtained solid residue was left to dry at room temperature (25° C.) for 24 hours.

Subsequently, the dried solid residue was subjected to solubilization in 30 chloroform (purity³ 99.8%—HiPerSolv CHROMANORM®—VWR) (ratio dried solid residue:chloroform equal to 1:200), at a temperature equal to 60° C., for 8 hours: said solubilization was repeated 3 times.

Subsequently, the whole was subjected to filtration through a porous septum filter obtaining a solid residue remaining on the filter, not comprising poly-3-hydroxybutyrate (PHB) and a solution comprising poly-3-hydroxybutyrate (PHB), which was placed in contact with an excess of ethanol (96°—Carlo Erba), at −20° C., for 10 minutes: the obtained precipitate comprising poly-3-hydroxybutyrate (PHB) was dried, at room temperature (25° C.), for 12 hours, giving a solid residue comprising poly-3-hydroxybutyrate (PHB).

The obtained solid residue comprising poly-3-hydroxybutyrate (PHB) was subjected to thermogravimetric analysis (TGA) according to the UNI EN ISO 11358-1:2014 TGA standard obtaining a solid residue, at 600° C., equal to 0.1% [solid residue not including poly-3-hydroxybutyrate—(PHB)].

The determination of the weight average molecular weight (M_{w}) of the obtained poly-3-hydroxybutyrate (PHB) was carried out by GPC (Gel Permeation Chromatography), operating as described above in Example 1 and it resulted to be equal to 1, 6×10⁶ Daltons.

The extraction yield of the whole process expressed as follows: grams of poly-3-hydroxybutyrate (PHB) obtained at the end of the process/grams of poly-3-hydroxybutyrate (PHB) synthesized from the microorganism used and available at the time of discharging from the fermenter was found to be >99%.

1. A process for recovering polyhydroxyalkanoate (PHA) from a fermentation broth deriving from the fermentation of microorganisms, the process having the following steps:
(a) subjecting the fermentation broth as such to heat treatment in the presence of at least one acid;
(b) subjecting the fermentation broth obtained in step (a), directly or after storage, to separation obtaining an aqueous suspension of cellular biomass comprising polyhydroxyalkanoate (PHA) and an aqueous phase comprising fermentation residues;
(c) subjecting the aqueous suspension of cellular biomass comprising polyhydroxyalkanoate (PHA) obtained in step (b) to extraction, purification and drying;
wherein said step (a) is carried out at a pH comprised between 4 and 6, at a temperature comprised between 50° C. and 70° C., for a time comprised between 5 minutes and 30 minutes.

2. The process for recovering polyhydroxyalkanoate (PHA) from a fermentation broth deriving from the fermentation of microorganisms according to claim 1, wherein said at least one acid is selected from inorganic acids or organic acids such as sulfuric acid, hydrochloric acid, acid nitric, phosphoric acid, acetic acid, citric acid.

3. The_Pprocess for recovering polyhydroxyalkanoate (PHA) from a fermentation broth deriving from the fermentation of microorganisms according to claim 1, wherein the fermentation broth obtained in step (a) is cooled to a temperature comprised between 5° C. and 40° C., in a time comprised between 5 minutes and 2 hours.

4. The process for recovering polyhydroxyalkanoate (PHA) from a fermentation broth deriving from the fermentation of microorganisms according to claim 1, wherein the fermentation broth obtained in step (a) is subjected to storage, for a time comprised between 4 hours and 24 hours.

5. The Process for recovering polyhydroxyalkanoate (PHA) from a fermentation broth deriving from the fermentation of microorganisms according to claim 1, wherein in said step (b) the fermentation broth obtained in step (a), directly or after storage, is subjected to tangential filtration and/or centrifugation, carried out in batch or continuously, obtaining an aqueous suspension of cellular biomass comprising polyhydroxyalkanoate (PHA) and an aqueous phase comprising fermentation residues.

6. The process for recovering polyhydroxyalkanoate (PHA) from a fermentation broth deriving from the fermentation of microorganisms according to claim 5, wherein at the end of said filtration and/or centrifugation, a cell concentration comprised between 200 g/l (dry weight) and 800 g/l (dry weight) is obtained.

7. The process for recovering polyhydroxyalkanoate (PHA) from a fermentation broth deriving from the fermentation of microorganisms according to claim 1, wherein said aqueous suspension of cellular biomass comprising polyhydroxyalkanoate (PHA), before being subjected to the step (c) of extraction, purification and drying, is subjected to a washing step by dilution with demineralized water or diluted aqueous solutions containing traces of cellular debris and subsequently, to a concentration step by tangential filtration and/or centrifugation, carried out in batch or continuously.

8. The process for recovering polyhydroxyalkanoate (PHA) from a fermentation broth deriving from the fermentation of microorganisms according to claim 7, wherein at the end of said washing step, a cell concentration comprised between 100 g/l (dry weight) and 400 g/l (dry weight) is obtained, while at the end of said concentration step a cell concentration comprised between 200 g/l (dry weight) and 800 g/l (dry weight) is obtained.

9. The process for recovering polyhydroxyalkanoate (PHA) from a fermentation broth deriving from the fermentation of microorganisms according to claim 1 wherein, in the event that said step (c) is carried out in the absence of solvent, the aqueous suspension of cellular biomass comprising polyhydroxyalkanoate (PHA) obtained in (b) of separation is subjected to mechanical and/or chemical cell lysis.

10. The process for recovering polyhydroxyalkanoate (PHA) from a fermentation broth deriving from the fermentation of microorganisms according to claim 9, wherein said aqueous suspension of cellular biomass comprising polyhydroxyalkanoate (PHA), is subjected to cell lysis by mechanical treatment, carried out using high pressure homogenizers, at a pressure comprised between 500 bar and 2000 bar, at a temperature comprised between 10° C. and 80° C.

11. The process for recovering polyhydroxyalkanoate (PHA) from a fermentation broth deriving from the fermentation of microorganisms according to claim 10, wherein at the end of said mechanical treatment, the obtained aqueous suspension comprising polyhydroxyalkanoate (PHA), is subjected to chemical cell lysis, said chemical cell lysis comprising:
basifying said aqueous suspension comprising polyhydroxyalkanoate (PHA) obtaining a pH value greater than or equal to 9, by adding a strong base, preferably an aqueous solution of at least one inorganic strong base such as sodium hydroxide, potassium hydroxide, or mixtures thereof obtaining a basified aqueous suspension comprising polyhydroxyalkanoate (PHA);
adding to said basified aqueous suspension comprising polyhydroxyalkanoate (PHA), at least one surfactant in an amount comprised between 0.05 g and 1 g, per gram of dry weight of said basified aqueous suspension comprising polyhydroxyalkanoate (PHA).

12. The process for recovering polyhydroxyalkanoate (PHA) from a fermentation broth deriving from the fermentation of microorganisms according to claim 11, wherein said chemical cell lysis is carried out at a temperature comprised between 10° C. and 80° C., for a time comprised between 20 minutes and 120 minutes.

13. The process for recovering and purifying polyhydroxyalkanoate (PHA) from a fermentation broth deriving from the fermentation of microorganisms according to claim 11, wherein the aqueous suspension comprising polyhydroxyalkanoate (PHA) obtained from chemical cell lysis, is subjected to a first concentration step by tangential filtration and/or centrifugation, carried out in batch or continuously, subsequently subjected to a washing step by dilution with demineralized water or diluted aqueous solutions containing traces of cellular debris, and subsequently subjected to a second concentration step by tangential filtration and/or centrifugation, carried out in batch or continuously.

14. The process for recovering polyhydroxyalkanoate (PHA) from a fermentation broth deriving from the fermentation of microorganisms according to claim 13, wherein at the end of said first concentration step a cell concentration comprised between 200 g/l (dry weight) and 800 g/l (dry weight), is obtained, at the end of said washing step, a cell concentration comprised between 100 g/l (dry weight) and 400 g/l, is obtained, and at the end of said second concentration step, a cell concentration comprised between 200 g/l (dry weight) and 800 g/l (dry weight), is obtained.

15. The process for recovering polyhydroxyalkanoate (PHA) from a fermentation broth deriving from the fermentation of microorganisms according to claim 11, wherein the aqueous suspension comprising polyhydroxyalkanoate (PHA) obtained from chemical cell lysis, is subjected to drying, at a temperature greater than or equal to 50° C., until a solid residue comprising polyhydroxyalkanoate (PHA) having a residual moisture of less than 0.5% is obtained.

16. The process for recovering polyhydroxyalkanoate (PHA) from a fermentation broth deriving from the fermentation of microorganisms according to claim 1 wherein, in the event that said step (c) is carried out in the presence of solvent, the aqueous suspension of cellular biomass comprising polyhydroxyalkanoate (PHA) obtained in step (b) of separation is subjected to freezing and freeze-drying.

17. The process for recovering polyhydroxyalkanoate (PHA) from a fermentation broth deriving from the fermentation of microorganisms according to claim 16, wherein the aqueous suspension of cellular biomass comprising polyhydroxyalkanoate (PHA) obtained in step (b) is subjected to a process comprising:
subjecting said aqueous suspension of cellular biomass comprising polyhydroxyalkanoate (PHA) to freezing, at −20° C., and subsequent freeze-drying carried out at −46° C., 0.15 mbar, for a time greater than or equal to 12 hours, until a dehydrated cellular powder comprising polyhydroxyalkanoate (PHA) is obtained;
subjecting the dehydrated cellular powder comprising polyhydroxyalkanoate (PHA) to washing with ethanol, at a temperature comprised between 30° C. and 60° C., for a time comprised between 30 minutes and 6 hours, by working at a dehydrated cellular powder:ethanol ratio comprised between 1:10 and 1:40, obtaining a suspension comprising polyhydroxyalkanoate (PHA);
filtering the obtained suspension comprising polyhydroxyalkanoate (PHA) with a porous septum filter or a cellulose filter and subjecting the solid residue comprising polyhydroxyalkanoate (PHA) left on the filter to drying, at room temperature (25° C.), for a time greater than or equal to 12 hours;
subjecting the obtained solid residue comprising dried polyhydroxyalkanoate (PHA) to solubilization in the presence of at least one solvent selected from chloroform, 1,2-dichloroethane, dichloromethane, in a stirred vessel or in a reflux reactor, operating at a dried solid residue:solvent ratio comprised between 1:100 and 1:500, for a time comprised between 4 hours and 24 hours, at a temperature comprised between 40° C. and 70° C. and, optionally, repeating said solubilization up to 3 times;
subjecting the solution comprising polyhydroxyalkanoate (PHA) obtained after solubilization to filtration with a septum filter or with a cellulose filter obtaining a solid residue free of polyhydroxyalkanoate (PHA) and a solution comprising polyhydroxyalkanoate (PHA);
optionally, concentrating the solution comprising polyhydroxyalkanoate (PHA) obtained by operating at a temperature lower than or equal to 70° C., at a pressure lower than atmospheric pressure;
placing the solution comprising polyhydroxyalkanoate (PHA), before or after any concentration, in contact with an excess of ethanol cooled to −20° C., in a stirred vessel, for a time comprised between 0.1 minutes and 60 minutes, obtaining the precipitation of a solid residue comprising polyhydroxyalkanoate (PHA);
mechanically collecting the obtained solid residue comprising polyhydroxyalkanoate (PHA) and subjecting it to drying at room temperature (25° C.), for a time greater than or equal to 12 hours, or, in a ventilated oven, at a temperature of 50° C., at low pressure, until a solid residue comprising polyhydroxyalkanoate (PHA) having a residual humidity lower than 0.5% is obtained.
